(19) United States
(12) Patent Application Publication
(10) Pub. No.: US 2008/0190432 A1
(43) Pub. Date: Aug. 14, 2008

(54) Mask Assembly

(75) Inventors: Fred Blochlinger, Victoria (AU); Warwick Freeman, Abbotsford Victoria (AU); Marina Omer-Cooper, Carnegie Victoria (AU)

Correspondence Address: SUGHRUE MION, PLLC, 2100 Pennsylvania Avenue, N.W., Washington, DC, 20037, US

(73) Assignee: Compumedics Limited, Abbotsford (AU)

(21) Appl. No.: 12/064,501

(22) PCT Filed: Aug. 21, 2006
(86) PCT No.: PCT/AU2006/001188

§ 371 Date: Mar. 13, 2008

(30) Foreign Application Priority Data
Aug. 22, 2005 (AU) 2005904530

(51) Int. Cl. A61M 16/06; A62B 18/02

(52) U.S. Cl. 128/205.25; 128/207.18; 128/207.13; 128/206.24

(57) ABSTRACT

The invention provides a nasal cushion for a mask assembly for delivering pressurised gas to the nose of a subject for treatment with continuous positive air pressure. The nasal cushion includes a self-supporting side wall defining a nose-receiving cavity; the side wall extending from a first end to a second end. The side wall is formed continuously with a thin membrane extending inwardly from a second end of the side wall into the nose-receiving cavity for sealingly engaging the nose, upper lip and cheeks of a subject. The side wall has continuously varying thickness. The first end of the side wall is adapted to engage a mask frame. The invention further provides a mask assembly for delivering pressurised gas to a patient, the assembly including a nasal cushion for sealing against the nose, cheek and-lip area the nasal cushion as described, a frame for receiving the nasal cushion, and a headgear, the headgear including a rigid arm for positioning behind the ear of a patient.

### FIELD OF THE INVENTION

The present invention relates to masks for the provision of gases to patients, in particular, CPAP and VPAP masks and mask assemblies.

### BACKGROUND

There are a number of treatments for patients that require the use of a mask for the delivery of gas, particularly air, to a patient's nose. For example, in the treatment of sleep apnoea, in particular, obstructive sleep apnoea (OSA) air is often delivered at continuous positive pressure (CPAP) wherein air is supplied continuously at a pressure greater than ambient, or variable positive pressure (VPAP) wherein air is supplied at varying pressures, to the nose of a sleeping patient through a mask to keep the patient's airways open for effective respiration. Often air must be delivered through a mask for sustained time periods, for example, through the whole overnight period of sleep.

It is important for continuous therapeutic benefit from pressurised air that a mask assembly be comfortable and relatively leak proof. A mask must maintain its position over the nose and seal with the face of a patient to maintain positive air pressure despite the movements of the patient during sleep that might break the seal. At the same time the mask must be comfortable so that a patient achieves the therapeutic benefit of relatively unbroken sleep periods.

Various mask assemblies have been developed to achieve adequate seals for the delivery of air under positive pressure to the nose of a patient. Prior art masks include “free-floating” nasal cushions for sealing against the face of a patient, i.e. the nasal cushions are not held in fixed position relative to the mask assembly of which they comprise a part, or fixed nasal cushions where the nasal cushion is fixed in place at a limited number of sealing positions. The fixed type of nasal cushion does not provide an effectively sealed and leak-proof mask for all users who have a broad range of facial features.

Masks are known in the art that incorporate a nasal cushion with two membranes, one for comfortable sealing against the face of a patient and another to provide comfortable but firm support against the face thereby ensuring that the seal is not broken and the CPAP effect is maintained.

Headgear is often comprised of a simple strap of an elastic material such as neoprene or similar material. Such a strap often causes discomfort to a patient during treatment. Such prior art mask assemblies usually are not held securely in place relative to the nose. The straps often stretch and slide around the head and over the hair allowing the nasal mask to move in relation to the wearer's face and breaking the air seal. Such movement can be very annoying to a patient even if the seal is not broken, resulting in a less effective treatment, particularly if the patient removes the mask because of the discomfort. The stretchable materials used in such mask assemblies have a limited lifespan in which they retain adequate elastic properties to achieve the desired sealing effect. This type of headgear can wear out relatively quickly.

Prior art masks are usually difficult to position on the face, especially by the wearer. In some known mask assemblies the headgear must be stretched over the head, which can be difficult or impossible for some patients, for example, elderly users, who often have reduced joint flexibility and strength to correctly position the mask assembly for treatment. Alternative mask assemblies in the art have headgear straps that must be completely loosened and re-tightened after correct positioning over the nose which is a tedious, difficult and time-consuming process. The removal of such assemblies can be very frustrating, leading the patient to stretch the headgear rather than loosening the fasteners. This process can be very painful to the patient, as well as reducing the effective life of the headgear. In such situations, to achieve a custom fit, the straps must be re-adjusted every time the mask is donned. Generally the mask assemblies in the art are a “one-size-fits-all” solution for a diverse group of patients requiring treatment. The masks cannot be precisely fine-tuned for a comfortable fit to suit the individual head dimensions and facial features of a patient once in position for treatment. Further, the manipulations of headgear described herein to achieve a correct positioning of a mask for treatment make it difficult to quickly remove and replace a mask from its proper treatment position on the head. The incorporation of engagement means such as buckles to hold together the straps of a headgear in prior art headgear presents a further problem. Buckles on the sides of headgear can decrease comfort during sleep if a user turns the head to result in pressure from a buckle on the side of the head or face.

What is needed is a CPAP/VPAP nasal cushion and mask assembly wherein the nasal cushion is simple to manufacture but it is easily inserted into the mask frame and removed from the mask frame for cleaning or replacement, for example, by a patient, particularly by a patient who has limited manual dexterity or strength. The mask assembly must also enable the nasal cushion to seal effectively to ensure a continuous positive air pressure supply to the patient during sleep. The mask assembly must include an effective headgear that keeps the nasal cushion in position and effectively sealed around the nose of a patient. The headgear must be simple to engage and disengage over the head of the patient while keeping the nasal cushion properly positioned over the nose. The headgear should be adjustable and customisable to achieve a comfortable, supportive fit for various users. Once headgear has been fitted and set, it should be easy to return the headgear to the set configuration, while still being easy to remove. Ideally, the headgear will incorporate features that allow a user to “set and forget” the adjustment of the various elements that achieve an effective yet comfortable fit.

### BRIEF DESCRIPTION OF THE DRAWINGS

FIG. 1 shows in side view a mask assembly in position for treatment on the head of a patient.

FIG. 2 shows in side view an exploded view of a mask assembly.

FIGS. 3a-c show in side view the operation of the headgear of the mask assembly in donning the mask on a patient's head for treatment.

FIGS. 4a-b shows in side view the engagement angles of the nasal cushion with the nose of a patient.

FIGS. 5a-b show in top view the nasal cushion engaging the nose of a patient.

FIG. 6 shows a nasal cushion and a mask frame in position for engagement.

FIGS. 7a and b show views of a nasal cushion of a mask.

FIG. 7c shows a clip for a nasal cushion in top view and side view.

### SUMMARY OF THE INVENTION

It is an object of the invention to provide a nasal cushion that is simple to manufacture but comfortable to wear but maintain a good seal for the effective delivery of CPAP. It will be understood that a reference to CPAP in this specification includes a reference to VPAP. It is a further object to provide a mask assembly that is easy to don but comfortable to wear for a subject while effectively providing CPAP treatment.

In one aspect, the invention provides a nasal cushion for a mask assembly for delivering pressurised gas to the nose of a subject, including a self-supporting side wall defining a nose-receiving cavity; said side wall extending from a first end to a second end; said side wall formed continuously with a thin membrane extending inwardly from a second end of said side wall into said nose-receiving cavity for sealingly engaging the nose, upper lip and cheeks of said subject; said side wall having continuously varying thickness; and said first end adapted to engage a mask frame. Preferably, the thin membrane has a variable width of extension into the nose-receiving cavity. The nasal cushion may include a nasal flap formed continuously with said thin membrane and further extending into said nose-receiving cavity for sealing engagement with the nose of said subject. The first end of the nasal may form a substantially flat surface. The side wall of the nasal cushion may include at least one thicker portion in at least one portion of the side wall along a longitudinal axis of the side wall, said thicker portion extending from the foot of the side wall. The nasal cushion may include a securing means for securing the nasal cushion to the nose of said subject. Preferably, the securing means includes an externally applied clip. Alternatively, the securing means may be over-moulded within said side wall. Preferably, the securing means is comprised of metal material.

In another aspect, the invention provides a mask assembly for delivering pressurised gas to a patient, the assembly including a nasal cushion for sealing against the nose, cheek and lip area the nasal cushion being as herein described, a frame for receiving the nasal cushion; and a headgear, the headgear including a rigid arm for positioning behind the ear of a patient. The mask assembly may comprise headgear including a cheekstrap, the cheekstrap including a pivotable engagement means for engaging the frame. The mask assembly may include a cheekstrap in the headgear, the cheekstrap including a positioning slot for positioning an engaging means for engaging the frame. The mask assembly may include the frame having a male engagement means for engaging said headgear.

### DETAILED DESCRIPTION OF THE DRAWINGS AND PREFERRED EMBODIMENTS

The invention provides a nasal cushion and mask assembly for CPAP treatment of a patient. It will be understood by a person skilled in the art that the examples of embodiments of the invention are illustrative and are not to be read as limiting the scope of the invention as it is defined by the appended claims. The mask assembly members advantageously offer many adjustment ranges and positioning angles that enable a highly customisable fit to accommodate variations in the dimensions of the heads and facial features of patients while providing effective gas delivery to the nose of a patient.

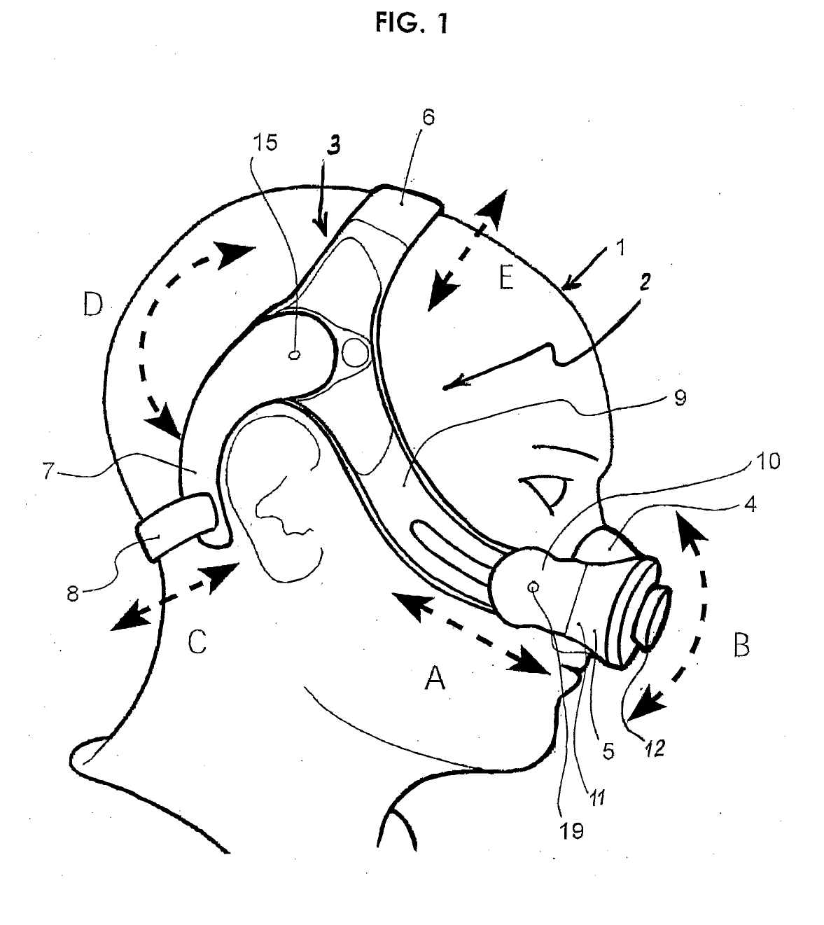

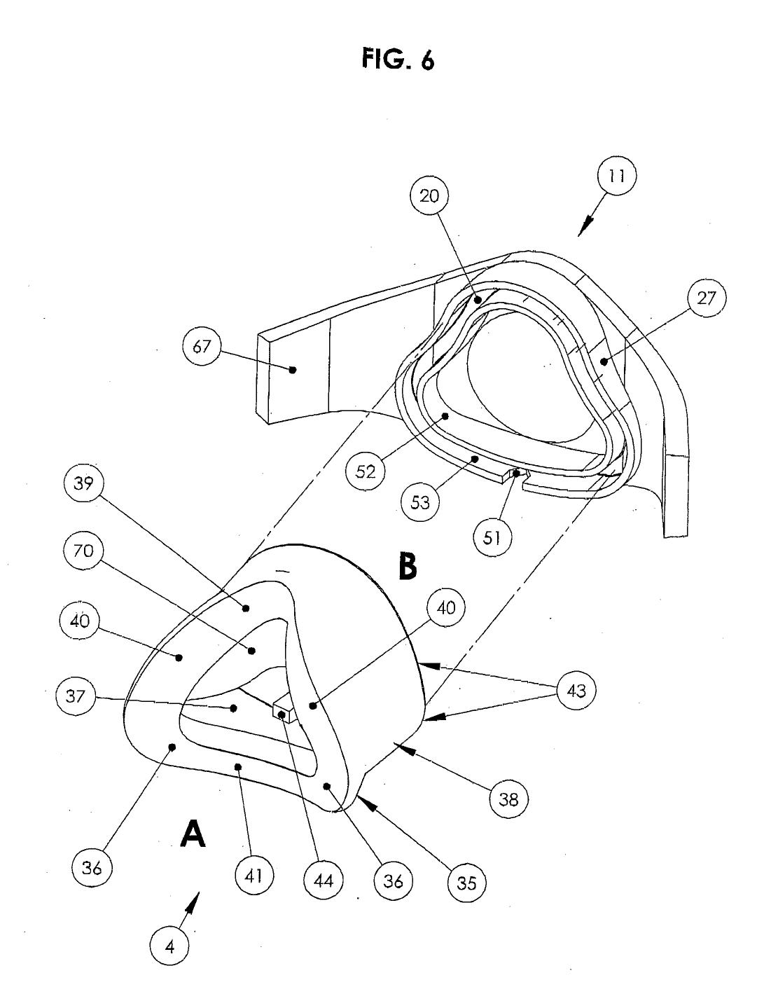

FIG. 1 shows in side perspective view a mask assembly 2 in position for treatment on the head of a patient 1. The mask assembly 2 comprises of headgear 3, nasal cushion 4, and mask frame 11, which cooperate to position the nasal cushion 4 around the nose of a patient 1 and to maintain its position during periods of treatment. The headgear 3 comprises of a headband 6, rotatable arms 7, neckstrap 8, and cheekstraps 9. The mask frame 11 may include a pivoting clip 10, receiving channel 20 (shown in FIG. 6) for the nasal cushion 4, and hose connector 12. The combination of features according to the invention allows adjustment of the mask assembly 2 in several different ways. It will be clear from the description herein that the invention advantageously provides several separately and cooperatively adjustable members of the mask assembly 2. The shape of patients' heads is variable and adjustment may be made in any or all of the adjustable members to provide a comfortable fit of the mask assembly 2 over the head of a patient 1, while positioning the nasal cushion 4 over the nose of the patient for effective treatment.

In FIG. 1, dashed lines indicate the direction of adjustment E of the headband 6, the direction of adjustment D of the rotatable arms 7, direction of adjustment C of the neckstrap 8, direction of adjustment A of a cheekstrap 9, and direction of adjustment B of mask frame 11. Individually, and in combination, adjustment of the adjustable elements operates to provide a comfortable fit of the mask assembly 2 over the head of a patient, while positioning the nasal cushion 4 over the nose of the patient for effective treatment. It will be understood that the figures provide only examples of the range of movement and that a comfortable and effective fit for treatment will be achieved by the patient in different ways in operation of the members of the invention.

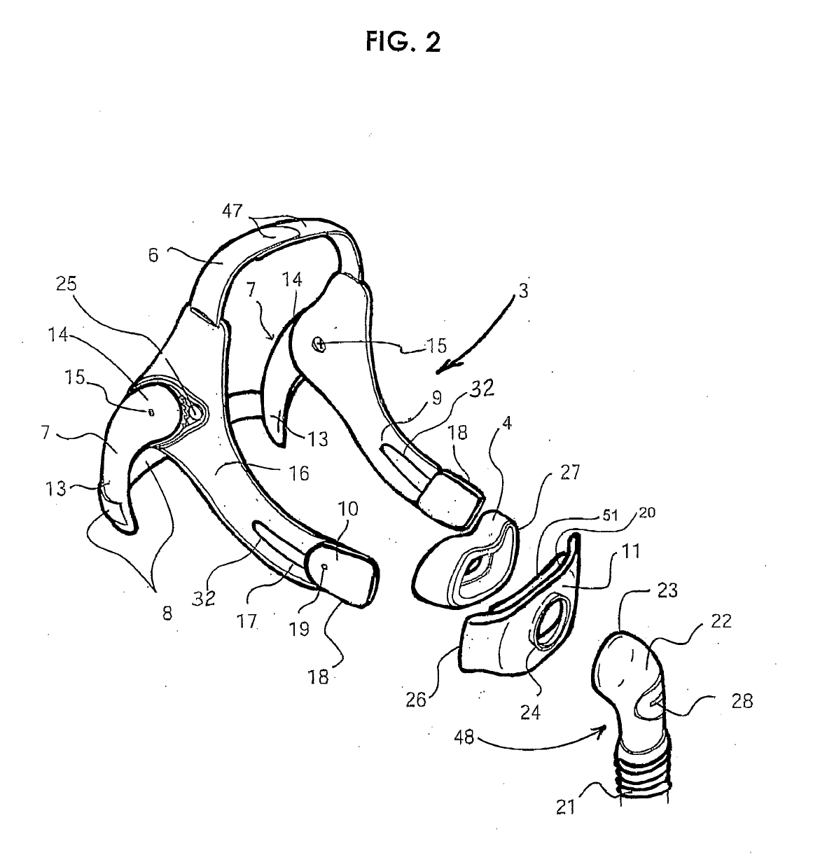

The headgear 3 includes three sets of straps as shown in FIGS. 1 to 4 as the straps 47 comprising the headband 6, the rotatable arm 7, the straps comprising the neckstrap 8 and the cheekstraps 9. In cooperative action said straps make the mask assembly 2 simple to don. The mask assembly 2 functions to provide a relatively leak-free conduit for the provision of gas to the nose of a patient by passage of the gas through the cooperation of a hose assembly 48 (shown in FIG. 2) and mask assembly 2. It will be understood by those skilled in the art that a reference to gas includes references to air, humidified air, or any other gas that is used in therapeutic treatment of a patient.

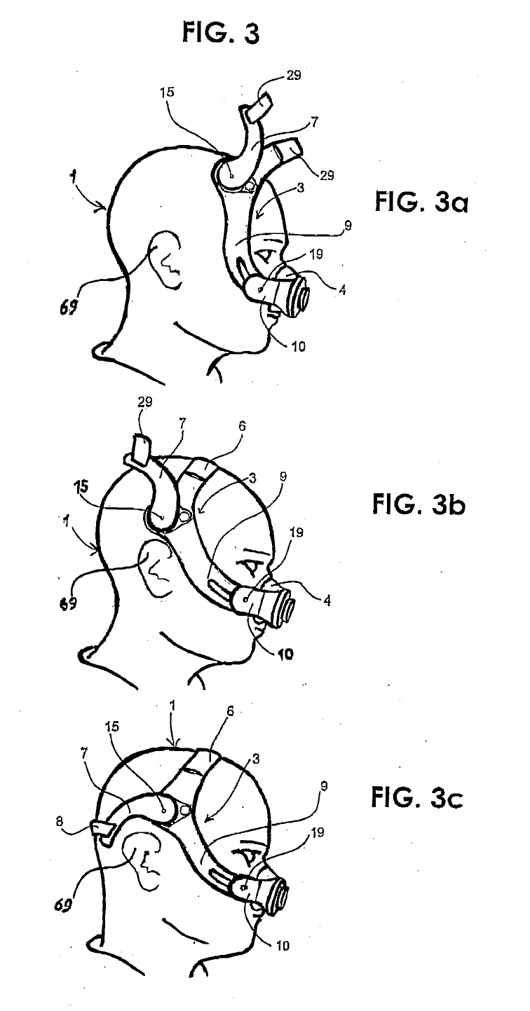

In FIGS. 3a-c is shown the wide direction of adjustment of the headgear 3 in the process of donning the mask assembly by a patient for treatment. The patient 1 in FIG. 3c has a different head shape from those in FIGS. 3a and 3b. The cooperation of the parts of the mask assembly described herein according to the invention provides an effective seal and comfortable fit for treatment for most head sizes and shapes. The invention provides the wide direction of adjustment by the pivoting of one or both of the rotatable arms 7 at an armature pivot point 15 and the pivoting of one or both of the cheekstraps 9 at the pivot point 19 of the pivoting clip 10. In FIG. 3a the neckstrap straps 29 are disengaged in the open position and the rotatable arms 7 are rotated away from the head of the patient 1. The cheekstraps 9 are pivoted upward at the pivot point 19 of the pivoting clip 10. The cooperative action at the two pivot points at each side of the mask assembly allows the headgear 3 to be swung away from the head of the patient to enable the correct positioning of nasal cushion 4. In FIG. 3b is shown an intermediate stage of donning the mask assembly 2 wherein the cheekstraps 9 and nasal cushion 4 are positioned for treatment while the rotatable arms 7 are moving towards the position to be achieved for treatment. In FIG. 3b it is shown that the members of the mask assembly 2 are in suitable positions for effective treatment when the mask assembly is fully donned.

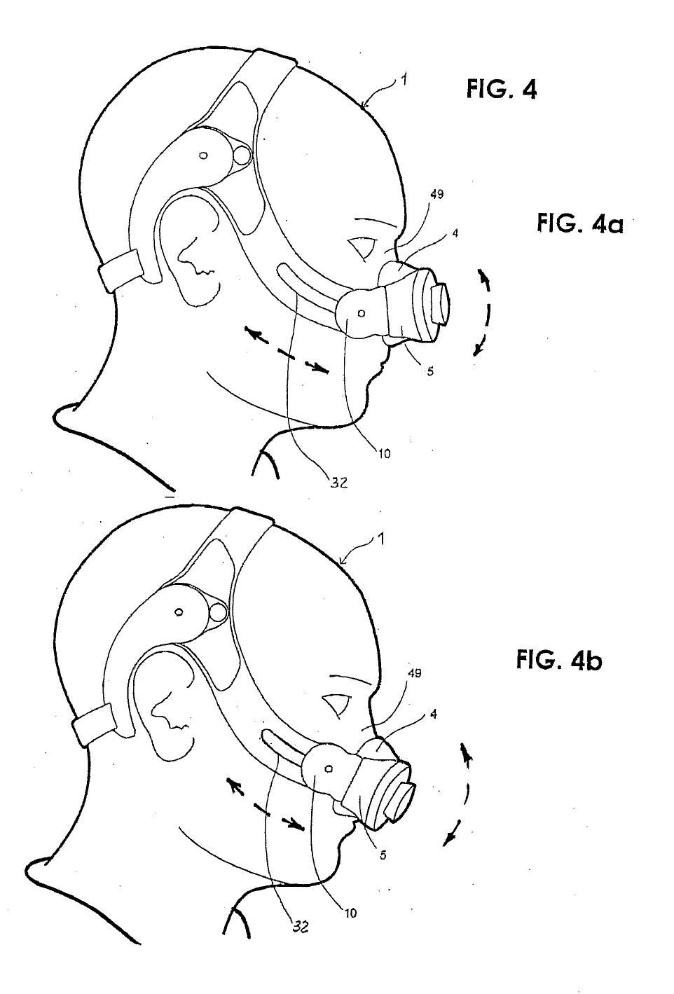

FIGS. 4a and 4b show the flexibility of orientation of the mask frame in relation to the nose of a patient once the mask assembly is donned by a patient 1. The flexibility of the adjustments described above allow for a range of “floating” movement of the mask frame 11. FIGS. 4a and 4b show a further aspect provided by the invention for patients with slightly different facial characteristics and structures, in particular, different lengths of the bridge of the nose 49. In FIG. 4a the mask frame 11 and nasal cushion 4 are oriented in a slight upward direction. The dashed lines in FIGS. 4a and 4b indicate that the mask frame and nasal cushion may not be fixed into position in some embodiments of the invention and there may be some degree of movement of the nasal cushion and mask frame by operation of the pivoting clip 10 while still retaining an effective seal, i.e. a free-floating nasal cushion is within the scope of the invention. In FIG. 4b the mask frame 11 and nasal cushion 4 are oriented in a slight downward direction. It will be understood that the operation of the mask assembly most advantageously provides an effective seal in these and other orientations within the large range or orientations of which the mask assembly is capable.

Consideration of the movement of the members of the mask assembly 2 in FIGS. 3a-c shows that an aspect of the invention provides that the members of the mask assembly may not engage with the ears 69 of the patient 1. This aspect of the invention provides a comfortable process of donning the mask assembly for the patient. A further aspect of the invention is that the movement of the adjustable members in the mask assembly is achieved with little force required to be applied by the patient. This is particularly advantageous for patients with restricted manual dexterity or strength. A further aspect and advantage provided by the invention is the ability to fix into any position either of the rotatable arms 7 at the armature pivot point or the pivot point of the pivoting clip 19. Fixing either of the members at an initial comfortable position provides for further convenience and comfort in positioning the mask assembly for treatment for later treatments.

The headband 6 is positioned over the top of a patient's head to assist in securing the nasal cushion over the nose of a patient 1. Preferably, the length of the headband 6 is adjustable by engagement of the headband straps 47 at different positions along the headband straps 47. The invention provides that engagement of the headband straps 47 may be achieved with any suitable means for secure engagement. Such means includes, but is not limited to, overlapping straps with hook and loop mechanical fasteners, fasteners including but not limited to, buttons or snaps, other mechanical fasteners known in the art, or buckles of any material. Preferably, the headband straps 47 are comprised of thin, flexible materials. Preferably, the material of which the headband straps 47 is comprised has some resilience so that patient comfort is ensured during long periods of wear.

A neckstrap 8 also assists in securing the nasal cushion 4 in cooperation with the adjustable headband 6 and cheekstraps 9. The neckstrap 8, shown in closed position in FIG. 2, engages with the distal ends 13 of rotatable arms 7, a rotatable arm 7 on either side of the patient's head, the proximal end 14 of each of the rotatable arms 7 including an armature pivotable about a pivot point 15 in its proximal end 14. The invention provides that engagement of the straps of the neckstrap 29 may be achieved with any suitable means for secure engagement. Such means includes, but is not limited to, overlapping straps with hook and loop mechanical fasteners, fasteners including but not limited to, buttons or snaps, other mechanical fasteners known in the art, or buckles of any material. Preferably, the straps of the neckstrap 29 are comprised of thin, flexible materials. Preferably, the material of which the straps of the neckstrap 29 are comprised has some resilience so that patient comfort is ensured during long periods of wear.

The rotatable arms 7 may be held in fixed position by the operation of an arm-lock means 25. The arm-lock means 25 may be comprised of any suitable engagement means which holds in fixed position the rotatable arms 7. The rotatable arms 7 may be comprised of a relatively rigid material. Preferably, the rotatable arms 7 are comprised of a rigid polymeric material such as plastic.

In one embodiment as shown in FIG. 2a, a cheekstrap 9 includes a distal end 16 for engaging the proximal ends of the rotatable arms 14 and a proximal end 17 to which is attached a pivoting clip 10 which contains a quick release clip 18. The quick release clip 18 has at one end a female receiving portion for engaging with the male engagement portion of the frame 26 for positioning the nasal cushion.

The cheekstraps 9 may be comprised of a relatively rigid material. Preferably, the cheekstraps 9 are comprised of a rigid polymeric material such as plastic.

The pivoting clip 10 conveniently pivots at a point of attachment 19 to the proximal end 17 of the cheekstrap 9. The pivoting clip may be engaged in fixed position by any suitable fixing means. The pivoting clip 10 and quick release clip 18 are comprised of a relatively rigid material. Preferably, the pivoting clip 10 and quick release clip are comprised of a rigid polymeric material such as nylon plastic. In operation the quick release clip 18 or the pivoting clip 10 at the proximal end 17 of a cheekstrap 9 on either side of the headgear 3 engages a receiving portion 26 of the rigid mask frame 11.

In FIGS. 4a and 4b is shown a further aspect provided by the invention in side perspective of a first side of the sides of the mask assembly 2 wherein the pivot clip is moveable along the positioning area 32. The pivoting clip 10 can be moved along the positioning area 32 in the proximal end of the cheekstrap 17. The invention provides most conveniently that the pivoting clip 10 can be positioned at a number of set locations in the positioning area and at a chosen angle to achieve a comfortable and effective fit for the nasal cushion 4. Alternatively, the pivoting clip 10 may not be in fixed position and may be allowed to pivot through a range in operation while the tension of the straps described herein maintains the nasal cushion in position for an effective seal. The invention provides that the pivoting clip 10 may be fixed at a suitable place for comfort of the patient while achieving effective treatment. In FIG. 4a the pivoting clip 10 is fixed at the proximal end 17 of the cheekstrap 9. In FIG. 4b the pivoting clip 10 is fixed away from the proximal end.

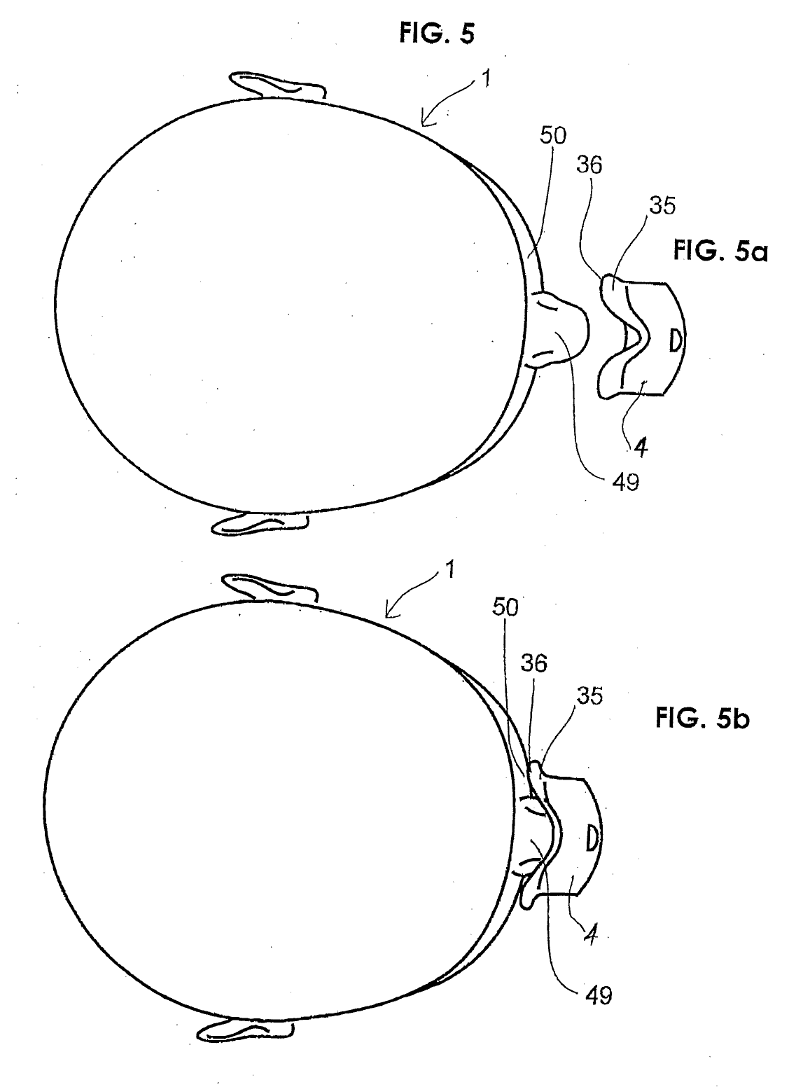

In FIG. 5 is shown in top view the operation of the mask assembly 2 to provide a seal between the nasal cushion 4 and the nose of a patient. Only the nasal cushion 4 of the mask assembly is shown. FIG. 5a shows the nasal cushion in correct orientation for engagement with the nose. FIG. 5b shows the nasal cushion engaged with the patient's nose during CPAP treatment. In FIG. 5b it is shown that the membrane and nose-engaging extension 39 of the nasal cushion 4 form a seal with the bridge of the nose 49 and cheeks 50 around the nose of the patient by deformation of the tapering flange 35 and membrane 36 of the nasal cushion 4. Similar deformation occurs at the upper lip of the patient (not shown). The deformation can be seen by comparing the tapering flange 35 and membrane 36 in FIG. 5a with those in FIG. 5b. In operation, as shown in FIGS. 5a and 5b, the flange 35 splays outwardly when pressed against a patient's face, conforming to a wide range of facial variations and providing spring-like support for the membrane 36 and nose-engaging extension 39, supports the membrane 36 and improves the seal of the membrane and nose-engaging extension with the patient's face to minimise air leakage. The resilience of the flange 35 and membrane 36 provides a comfortable seal even while a patient moves around while sleeping.

Nasal Cushion

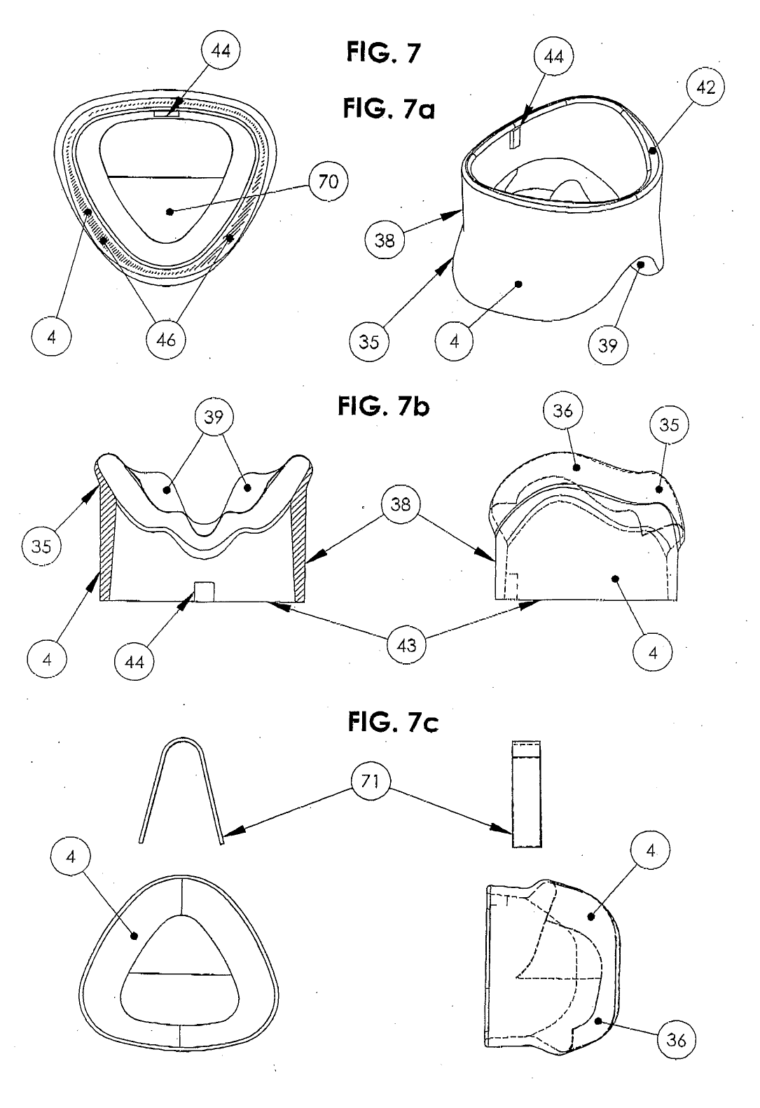

The invention provides a nasal cushion with a membrane forming a generally trilobular edge that effectively seals around the nose and upper lip of a patient to maintain a pressure at the nose of the patient when gas is blown into the nasal cushion. The nasal cushion is mostly conveniently made of resilient, biocompatible, pliable, tear-resistant elastomeric material. Embodiments of nasal cushions according to the invention are shown in FIGS. 6 and 7. The shape of the nasal cushion 4 has two distinct areas: the side wall of ever-changing thickness of the supportive body 38, and a thin-walled membrane 36 with nose-engaging extension 39, that may include a nasal flap 70, that forms the nose-receiving cavity 37 for conforming to the patient's face to provide an air seal. The side wall 38 includes a portion that operates as a flange 35 which collapses in a controlled way as described herein under headgear strap pressure. The patient's nose is inserted through the nasal opening 37 at side A, as shown in FIGS. 5a and 5b. Also shown in FIG. 6 is the mask frame 11 into a concave side of which is inserted the engagement edge 43 of the nasal cushion 4. In FIG. 7a is shown a bottom view of the nasal cushion 4. The thin-walled membrane 36 is very advantageously shaped to have a nose-engaging section 39, a cheek-engaging section 40, and a lip-engaging section 41. Each of these sections preferably extends from the flange for different distances, to comprise a membrane with variable width to provide a secure but comfortable seal in operation of the mask assembly. The hatched area indicates the foot 42 of the nasal cushion 4 which engages the mask frame 11 at the engagement edge 43. It will be understood that the invention provides nasal cushions of different sizes and dimensions suitable for engagement with the noses of patients with a wide range of facial features.

The side wall 38 extends substantially perpendicularly from the engagement edge 43 that engages the mask frame 11. In top view the side wall forms an outward extending curve. A distal portion of the side wall 38 outwardly flares to form a flange 35 in sealing with the nose and face of a patient. It can be seen in FIGS. 7b and 7c that the side wall 38 has a continuously varying thickness, shown in transverse section as the hatched area in FIG. 7b. The hatched area indicates that the engagement end 43 of the side wall 38 is a first thickness in cross section, increasing in thickness to a portion near the flange 35 and the flange is continuous with the side wall 38, extending from the side wall and decreasing in thickness. Continuous with the flange 35 and extending into the nose-receiving cavity 37 is a very thin membrane 36 and nose-engaging extension 39 for engaging sealingly with the face of patient. Preferably, the thin membrane 36 includes a nasal flap 70 for enhanced sealing engagement with the nose and face of a patient. The membrane 36 curves gently towards the side wall 38 to provide added comfort to the patient.

The extension of the membrane from the flange 35 is variable, for example, it may be less at a portion 39 of the side wall where the membrane is curved to receive the nose of a patient to provide an air seal for a greater range of membrane travel allowing the membrane to provide an effective seal for a greater range of patient nose bridge widths and depths. Similarly, the extension of the membrane is less at a portion 41 for sealing engagement with the upper lip. It will be understood that the figures provided herein are exemplary only of embodiments of the invention and that the width of the membrane 36 extending from the tapering flange 35 may be variable in different areas of the membrane. Alternatively, the thin membrane 36 may include a nasal flap 70 as an extension of the nasal engaging portion 39.

As shown in FIG. 7a, a portion 46 of the foot 42 is thickened in cross-section relative to the remainder of the foot 42 to provide reinforcement of the side wall near the patient's eyes, where the patient's nose meets the cheek. A thickened portion of the foot is a thickening in a dimension substantially perpendicular to the varying thickness from the thickening of the side wall 38 as the side wall extends from the engagement end 43 to the flange 35. This extra thickness in the dimension generally parallel with the engagement end 43 provides extra sealing force in a critical and difficult sealing area. It will be understood that the invention is not limited to a single portion of thickened area and that other portions of the foot 42 and side wall 38 may be thickened in other areas to optimise the ability of the side wall to provide support for the sealing of the membrane of the nasal mask against the face of a patient while providing a comfortable fit for the patient.

The continuous membrane 36 over the nose bridge including the nose engagement portion 39 provides minimal leakage of gas from the nasal cushion in the areas around the nose and under the eyes in which leaks are most likely to occur and to be annoying to a patient during treatment. The nose flap 70 functions to increase the surface area of the membrane 36 to improve the effectiveness of the seal between the face of the patient and the nasal cushion. In operation of a gas delivery device in communication with the nasal cushion, the supply of gas under pressure to the aperture of the nasal cushion causes the nose flap 70 to mould to the facial features of the patient to cooperate with the other portions of the thin membrane 36 to achieve an enhanced seal.

In FIG. 7c is shown a further embodiment of the nasal cushion, including a securing means 71 to engage the nasal cushion firmly with the bridge of the nose of a patient. Preferably the securing means 71 is a clip. Preferably the securing means is constructed of a resilient material that is capable of shape retention, such as a metal material. The securing means is shown in FIG. 7c as an externally applied clip. Alternatively, it make take an embodiment such as being embedded in the side wall 38 of the nasal cushion 4 in the area of the side wall near the nose-engaging section 39. Preferably the securing means is over-moulded within the side wall 38.

Mask Frame

As shown in FIG. 6, the rigid mask frame 11 receives the nasal cushion 4 into a receiving channel 20 on the inside of the mask frame 11, the receiving channel 20 holding the nasal cushion 4 in fixed orientation so that the nasal cushion opening 37 sealingly engages the nose cheeks and upper lip of the patient when the mask assembly is in place for treatment. The edge of engagement of the nasal cushion 43 is inserted into the receiving channel 20 of the mask frame 11 in movement generally along the dashed lines. An orientation tab 44 engages with the cut-out 51 to assist a user to properly orient the nasal cushion for engagement in fixed position in the mask frame 11.

A flexible air hose 21 engages with a rotating hose connector elbow joint 22. The connector elbow joint 22 may rotate to allow greater flexibility of movement. An end of the elbow joint 23 engages with a mask frame hose connector 24 located on the outside surface of the rigid mask frame 11. The hose connector elbow joint 22 may contain an exhaust air port or ports 28 for expired gases. Preferably the mask frame 11 is rigid injection moulded plastic.

1. A nasal cushion for a mask assembly for delivering pressurised gas to the nose of a subject, including;
a self-supporting side wall defining a nose-receiving cavity;
said side wall extending from a first end to a second end;
said side wall formed continuously with a thin membrane extending inwardly from a second end of said side wall into said nose-receiving cavity for sealingly engaging nose, upper lip and cheeks of said subject;
said side wall having continuously varying thickness; and
said first end adapted to engage a mask frame.

2. The nasal cushion according to claim 1, wherein the thin membrane has a variable width of extension into said nose-receiving cavity.

3. The nasal cushion according to claim 2, further including a nasal flap formed continuously with said thin membrane and further extending into said nose-receiving cavity for sealing engagement with the nose of said subject.

4. The nasal cushion according to claim 3 wherein said first end forms a substantially flat surface.

5. The nasal cushion according to claim 4, wherein said side wall includes at least one thicker portion in at least one portion of the side wall along a longitudinal axis of the side wall, said thicker portion extending from the foot of the side wall.

6. The nasal cushion according to claim 5 further including a securing means for securing the nasal cushion to the nose of said subject.

7. The nasal cushion according to claim 6 wherein the securing means includes an externally applied clip.

8. The nasal cushion according to claim 7 wherein the securing means is over-moulded within the said side wall.

9. The nasal cushion according to claim 8 wherein the securing means is comprised of metal material.

10. A mask assembly for delivering pressurized gas to a patient, the assembly including:
a nasal cushion for sealing against the nose, cheek and lip area, wherein the nasal cushion further comprises:
a self-supporting side wall defining a nose-receiving cavity;
said side wall extending from a first end to a second end;
said side wall formed continuously with a thin membrane extending inwardly from a second end of said side wall into said nose-receiving cavity for sealingly engaging nose, upper lip and cheeks of said subject;
said side wall having continuously varying thickness; and
said first end adapted to engage a mask frame;
a frame for receiving the nasal cushion; and
a headgear;
the headgear including a rigid arm for positioning behind the ear of a patient.

11. The nasal cushion according to claim 10, wherein the thin membrane has a variable width of extension into said nose-receiving cavity.

12. The nasal cushion according to claim 11, further including a nasal flap formed continuously with said thin membrane and further extending into said nose-receiving cavity for sealing engagement with the nose of said subject.

13. The nasal cushion according claim 12 wherein said first end forms a substantially flat surface.

14. The nasal cushion according claim 13, wherein said side wall includes at least one thicker portion in at least one portion of the side wall along a longitudinal axis of the side wall, said thicker portion extending from the foot of the side wall.

15. The nasal cushion according to claim 14 further including a securing means for securing the nasal cushion to the nose of said subject.

16. The nasal cushion according to claim 15 wherein the securing means includes an externally applied clip.

17. The nasal cushion according to claim 16 wherein the securing means is over-moulded within the said side wall.

18. The nasal cushion according to claim 17 wherein the securing means is comprised of metal material.

19. The mask assembly of claim 18, said headgear including a cheekstrap, the cheekstrap including a pivotable engagement means for engaging the frame.

20. The mask assembly of claim 19, further including a cheekstrap in the headgear, the cheekstrap including a positioning slot for positioning an engaging means for engaging the frame.

21. The mask assembly of claim 20, the frame having a male engagement means for engaging said headgear.
